(19) United States
(12) Patent Application Publication
(10) Pub. No.: US 2004/0230201 A1
(43) Pub. Date: Nov. 18, 2004

(54) Prostheses, tools and methods for replacement of natural facet joints with artifical facet joint surfaces

(75) Inventors: Hansen A. Yuan, Fayetteville, NY (US); David Stinson, Woodinville, WA (US); Lawrence R. Jones, Conifer, CO (US); Mark A. Reiley, Piedmont, CA (US)

Correspondence Address: WILSON SONSINI GOODRICH & ROSATI, 650 PAGE MILL ROAD, PALO ALTO, CA, 943041050

(73) Assignee: Archus Orthopedics Inc.

(21) Appl. No.: 10/438,295

(22) Filed: May 14, 2003

(51) Int. Cl. A61B 17/58

(52) U.S. Cl. 606/99

(57) ABSTRACT

Tools for installing artificial cephalad and caudal vertebral facet joint prostheses are provided. Specifically, an installation fixture for aligning and installing caudal, cephalad, or caudal and cephalad facet joint prostheses is provided. Also provided is a guide tool for use with the installation fixture for guiding the affixation of a prosthesis to a vertebra.

### FIELD OF THE INVENTION

This invention relates to prostheses for treating various types of spinal pathologies, as well as to methods of treating spinal pathologies.

### BACKGROUND OF THE INVENTION

I. Vertebral Anatomy

As FIG. 1 shows, the human spinal column 10 is comprised of a series of thirty-three stacked vertebrae 12 divided into five regions. The cervical region includes seven vertebrae 12, known as C1-C7. The thoracic region includes twelve vertebrae 12, known as T1-T12. The lumbar region contains five vertebrae 12, known as L1-L5. The sacral region is comprised of five vertebrae 12, known as S1-S5. The coccygeal region contains four vertebrae 12, known as Co1-Co4.

FIG. 2 shows a normal human lumbar vertebra 12. Although the lumbar vertebrae 12 vary somewhat according to location, they share many features common to most vertebrae 12. Each vertebra 12 includes a vertebral body 14 and posterior elements as follows:

Two short bones, the pedicles 16, extend backward from each side of the vertebral body 14 to form a vertebral arch 18. At the posterior end of each pedicle 16 the vertebral arch 18 flares out into broad plates of bone known as the laminae 20. The laminae 20 fuse with each other to form a spinous process 22. The spinous process 22 serves for muscle and ligamentous attachment. A smooth transition from the pedicles 16 into the laminae 20 is interrupted by the formation of a series of processes.

Two transverse processes 24 thrust out laterally on each side from the junction of the pedicle 16 with the lamina 20. The transverse processes 24 serve as levers for the attachment of muscles to the vertebrae 12. Four articular processes, two superior 26 and two inferior 28, also rise from the junctions of the pedicles 16 and the laminae 20. The superior articular processes 26 are sharp oval plates of bone rising upward on each side from the union of the pedicle 16 with the lamina 20. The inferior processes 28 are oval plates of bone that extend in an inferior direction on each side.

The superior and inferior articular processes 26 and 28 each have a natural bony structure known as a facet. The superior articular facet 30 faces upward or superiorly, while the inferior articular facet 31 faces downward. As FIG. 3 shows, when adjacent (i.e., cephalad and caudal) vertebrae 12 are aligned, the facets 30 and 31, capped with a smooth articular cartilage, interlock to form a facet joint 32, also known as a zygapophysial joint.

The facet joint 32 is composed of a superior half and an inferior half. The superior half is formed by the vertebral level below the joint 32, and the inferior half is formed by the vertebral level above the joint 32. For example, in the L4-L5 facet joint, the superior portion of the joint is formed by bony structure on the L-5 vertebra (e.g., a superior articular surface and supporting bone on the L-5 vertebra), and the inferior portion of the joint is formed by bony structure on the L-4 vertebra (e.g., an inferior articular surface and supporting bone on the L-4 vertebra).

As also shown in FIG. 3, an intervertebral disc 34 between each pair of vertebrae 12 permits relative movement between vertebrae 12. Thus, the structure and alignment of the vertebrae 12 permit a range of movement of the vertebrae 12 relative to each other.

II. Facet Joint Dysfunction

Back pain, particularly in the “small of the back”, or lumbosacral (L4-S1) region, is a common ailment. In many cases, the pain severely limits a person's functional ability and quality of life. Such pain can result from a variety of spinal pathologies.

Through disease or injury, the laminae, spinous process, articular processes, or facets of one or more vertebrae can become damaged, such that the vertebrae no longer articulate or properly align with each other. This can result in an undesired anatomy, pain or discomfort, and loss of mobility.

For example, the vertebral facet joints can be damaged by either traumatic injury or by various disease processes. These disease processes include osteoarthritis, ankylosing spondylolysis, and degenerative spondylolisthesis. The damage to the facet joints often results in pressure on nerves, also called a “pinched” nerve, or nerve compression or impingement. The result is pain, misaligned anatomy, and a corresponding loss of mobility. Pressure on nerves can also occur without facet joint pathology, e.g., a herniated disc.

One type of conventional treatment of facet joint pathology is spinal stabilization, also known as intervertebral stabilization. Intervertebral stabilization prevents relative motion between the vertebrae. By preventing movement, pain can be reduced. Stabilization can be accomplished by various methods.

One method of stabilization is posterior spinal fusion. Another method of stabilization is anterior spinal fusion, fixation of any number of vertebrae to stabilize and prevent movement of the vertebrae.

Another type of conventional treatment is decompressive laminectomy. This procedure involves excision of the laminae to relieve compression of nerves.

These traditional treatments are subject to a variety of limitations and varying success rates. Furthermore, none of the described treatments puts the spine in proper alignment or return the spine to a desired anatomy. In addition, stabilization techniques, by holding the vertebrae in a fixed position, permanently limit the relative motion of the vertebrae, altering spine biomechanics.

### SUMMARY OF THE INVENTION

There is a need for prostheses, installation tools, and methods that overcome the problems and disadvantages associated with current strategies and designs in various treatments for spine pathologies.

The invention provides prostheses, installation tools, and methods designed to replace natural facet joints at virtually all spinal levels including L1-L2, L2-L3, L3-L4, L4-L5, L5-S1, T-11-T12, and T12-L1. The prostheses, installation tools, and methods can restore a desired anatomy to a spine and give back to an individual a desired range of relative vertebral motion. The prostheses, installation tools, and methods also can lessen or alleviate spinal pain by relieving the source of nerve compression or impingement.

For the sake of description, the prostheses that embody features of the invention will be called either “cephalad” or “caudal” with relation to the portion of a given natural facet joint they replace. As previously described, a given natural facet joint has a superior half and an inferior half. In anatomical terms, the superior half of the joint is formed by the vertebral level below the joint (which can thus be called the caudal portion of the facet joint, i.e., because it is near the feet). The inferior half of the joint is formed by the vertebral level above the joint (which can thus be called the cephalad portion of the facet joint, i.e., because it is near the head). Thus, a prosthesis that, in use, replaces the caudal portion of a facet joint (i.e., the superior half) will be called a “caudal” prosthesis. Likewise, a prosthesis that, in use, replaces the cephalad portion of a facet joint (i.e., the inferior half) will be called a “cephalad” prosthesis.

One aspect of the invention provides an installation tool for implanting a prosthesis assembly. According to this aspect of the invention, the tool includes right and left caudal facet joint bearing element attachment mechanisms; and a caudal facet joint bearing element spacing adjuster (such as an actuator) adapted to change the spacing between right and left caudal facet joint bearing element attachment mechanisms. The tool may include caudal and cephalad facet joint bearing element alignment elements adapted and configured to mate with corresponding alignment elements on facet joint bearing elements. The installation tool may also include right and left cephalad facet joint bearing element attachment mechanisms oriented with the right and left caudal facet joint bearing element attachment mechanisms such that a right cephalad facet joint bearing element mounted in the right cephalad facet joint bearing element attachment mechanism aligns with a right caudal facet joint bearing element mounted in the right caudal facet joint bearing element attachment mechanism and a left cephalad facet joint bearing element mounted in the left facet joint bearing element attachment mechanism aligns with a left caudal facet joint bearing element mounted in the left caudal facet joint bearing element attachment mechanism. The installation tool may also include an insertion path guide interface adapted and configured to orient a fixation element insertion path guide in a specific orientation with respect to at least one of the right and left cephalad facet joint bearing surfaces. The insertion path guide interface may also be adapted and configured to orient a fixation element insertion path guide to guide the attachment of a cephalad facet joint bearing element to a vertebra without blocking access to a pedicle portion of the vertebra.

Another aspect of the invention provides a tool for installing a facet joint prosthesis. According to this aspect of the invention, the tool includes a caudal facet joint bearing surface attachment mechanism; a cephalad facet joint bearing surface attachment mechanism adapted and configured to align a cephalad joint bearing surface with a caudal facet joint bearing surface mounted in the caudal facet joint bearing surface attachment mechanism; and an insertion path guide interface. The insertion path guide interface may be adapted and configured to orient a fixation element insertion path guide in a specific orientation with respect to a cephalad facet joint bearing surface. The insertion path guide interface may also be adapted and configured to orient a fixation element insertion path guide to guide the orientation of a cephalad facet joint fixation element in a lamina portion of a vertebra. The insertion path guide interface may also be adapted and configured to orient a fixation element insertion path guide to guide the attachment of a cephalad facet joint bearing element to a vertebra without blocking access to a pedicle portion of the vertebra. The caudal facet joint bearing surface attachment mechanism and the cephalad facet joint bearing surface attachment mechanism may also be further adapted and configured to place the caudal facet joint bearing surface and the cephalad facet joint bearing surface in compression.

Another aspect of the invention provides a guide tool for guiding the insertion path of a fixation element for a cephalad facet joint bearing element of a facet joint prosthesis. According to this aspect of the invention, the guide tool includes a handle; a facet joint bearing element alignment interface; and an insertion path guide surface. The guide tool may include an arm extending from the handle and being adapted and configured to orient the insertion path guide surface along an axis extending through a lamina portion of a vertebra.

Another aspect of the invention is a method for implanting a cephalad facet joint prosthesis to replace a removed cephalad portion of a natural facet joint on a vertebra. This method includes the steps of placing the cephalad facet joint prosthesis in a desired position with respect to the vertebra; aligning a guide tool with the cephalad facet joint prosthesis; and using the guide tool to guide an insertion path for a cephalad facet joint fixation element. In the method, the placing step may include placing the cephalad facet joint prosthesis between a caudal facet joint bearing surface and the vertebra, and the aligning step may include aligning a guide tool alignment interface with cephalad facet joint prosthesis. The method may also include the step of forming a passage in the vertebra, such as by drilling, and the using step may include the step of inserting the cephalad facet joint fixation element, such as through a lamina portion of the vertebra.

Yet another aspect of the invention provides a facet joint prosthesis installation assembly. According to this aspect of the invention, the installation assembly includes a caudal facet joint bearing surface attachment mechanism; a cephalad facet joint bearing surface attachment mechanism adapted and configured to align a cephalad joint bearing surface with a caudal facet joint bearing surface mounted in the caudal facet joint bearing surface attachment mechanism; a guide tool for guiding the insertion path of a fixation element for a cephalad facet joint bearing surface of the facet joint prosthesis; and a guide tool interface adapted and configured to orient the guide tool to guide the insertion path of a cephalad facet joint bearing surface fixation element in a vertebra.

Other features and advantages of the inventions are set forth in the following Description and Drawings, as well as in the appended claims.

### DESCRIPTION OF THE DRAWINGS

FIG. 1 is a lateral elevation view of a normal human spinal column;

FIG. 2 is a superior view of a normal human lumbar vertebra;

FIG. 3 is a lateral elevation view of a vertebral lumbar facet joint;

FIG. 4 is a posterior view of an artificial facet joint prosthesis installed in a patient according to one embodiment of this invention;

FIG. 5 is a left side view of the embodiment of FIG. 4, as installed in a patient;

FIG. 6 is yet another view of the embodiment of FIG. 4, as installed in a patient;

FIG. 7A is a cross-sectional view of a cephalad bearing element and fixation element according to the embodiment of FIG. 4;

FIG. 7B is a posterior view of a pair of artificial cephalad and caudal facet joint prostheses according to one embodiment of this invention;

FIG. 7C is a top view of a pair of artificial cephalad and caudal facet joint prostheses in the embodiment of FIG. 7A;

FIG. 7D is a left view of a pair of artificial cephalad and caudal facet joint prostheses in the embodiment of FIG. 7A;

FIG. 7E is a bottom view of a pair of artificial cephalad and caudal facet joint prostheses in the embodiment of FIG. 7A;

FIG. 7F is an anterior view of a pair of artificial cephalad and caudal facet joint prostheses in the embodiment of FIG. 7A;

FIG. 8A is a perspective view of an installation fixture according to one embodiment of this invention;

FIG. 8B is a top view of the installation fixture of FIG. 8A;

FIG. 8C is a side view of the installation fixture of FIG. 8A;

FIG. 8D is a back view of the installation fixture of FIG. 8A;

FIG. 9 is an exploded view of the installation fixture of FIG. 8 along with a pair of caudal facet bearing elements and a pair of cephalad facet bearing elements according to one embodiment of the invention;

FIGS. 10A-D are views of a guide tool according to one embodiment of the invention;

FIG. 11 is a posterior view of the installation fixture of FIGS. 8 and 9 to which a pair of caudal facet bearing elements and a pair of cephalad bearing elements have been attached and with the caudal bearing elements attached to the patient;

FIG. 12 is a left side view of the installation fixture and bearing elements of FIG. 11 with the caudal bearing elements attached to the patient;

FIG. 13 is a perspective view of the installation fixture and bearing elements of FIGS. 11 and 12 showing a guide tool according to one embodiment of this invention;

FIG. 14 is a perspective view of the installation fixture and bearing elements of FIGS. 11 and 12 showing the use of a drill bit with the guide tool according to one embodiment of this invention.

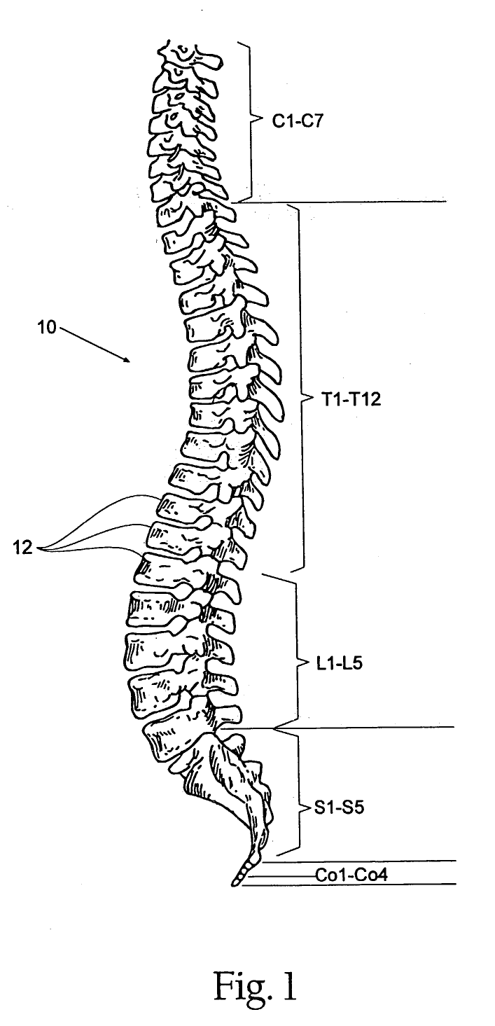

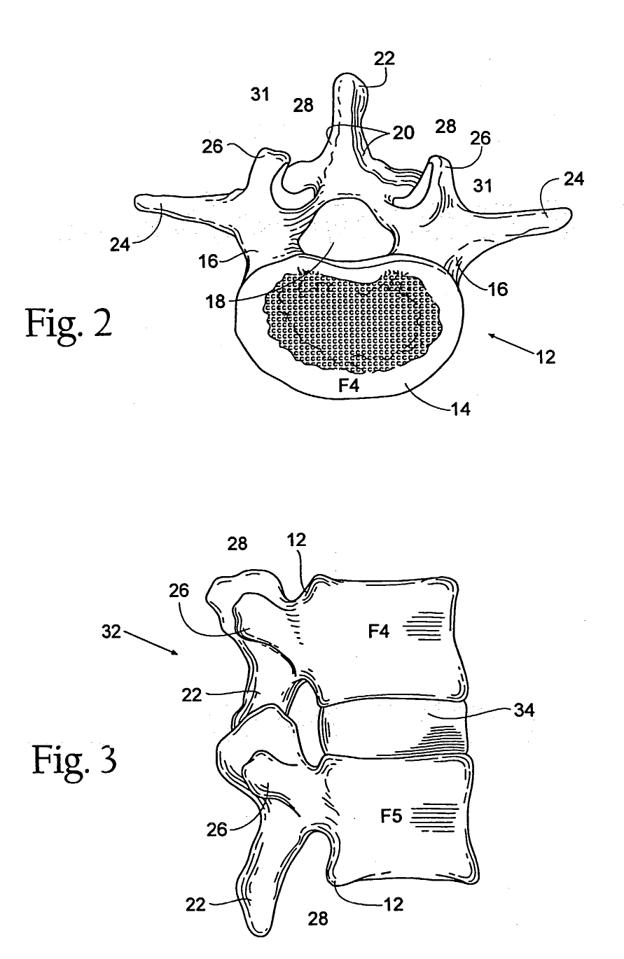

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION

Although the disclosure hereof is detailed and exact to enable those skilled in the art to practice the invention, the physical embodiments herein disclosed merely exemplify the invention that may be embodied in other specific structure. While the preferred embodiment has been described, the details may be changed without departing from the invention, which is defined by the claims.

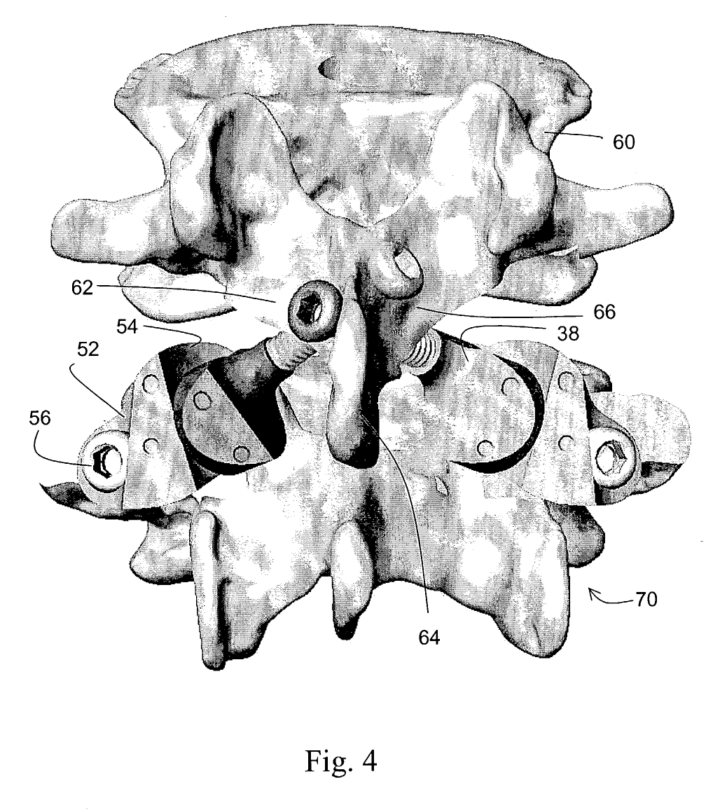

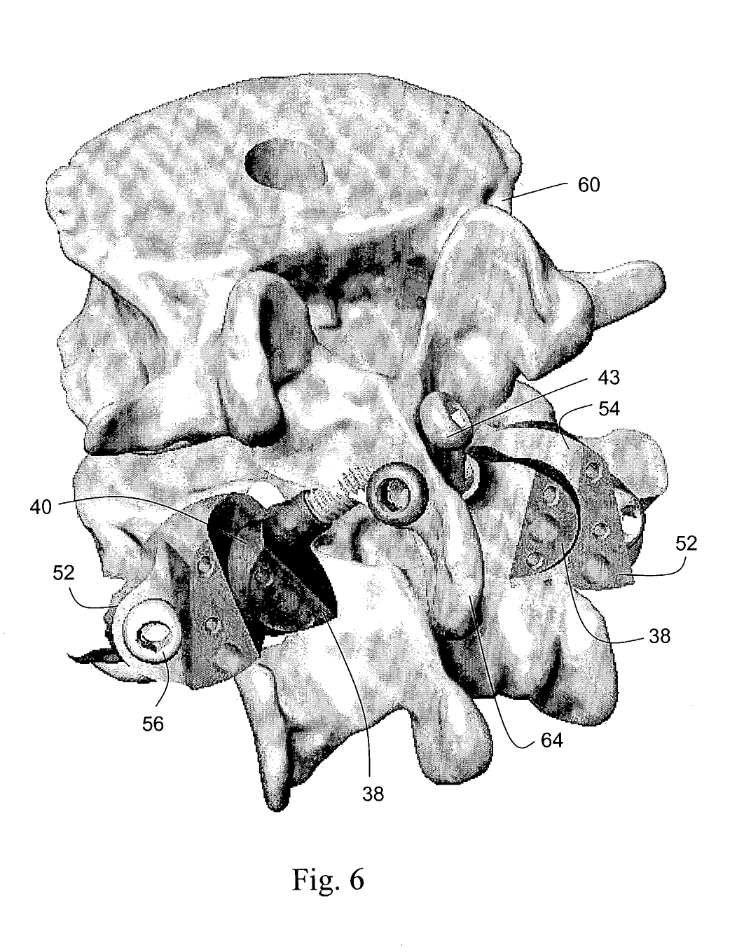

FIGS. 4-7 show artificial cephalad and caudal facet joint prostheses 36 and 50 for replacing a natural facet joint according to one aspect of this invention. Cephalad prosthesis 36 has a bearing element 38 with a bearing surface 40. In this embodiment, bearing surface 40 has a convex shape. Bearing element 38 may be formed from biocompatible metals (such as cobalt chromium steel, surgical steels, titanium, titanium alloys, tantalum, tantalum alloys, aluminum, etc.), ceramics, polyethylene, biocompatible polymers, and other materials known in the prosthetic arts, and bearing surface 40 may be formed from biocompatible metals (such as cobalt chromium steel, surgical steels, titanium, titanium alloys, tantalum, tantalum alloys, aluminum, etc.), ceramics, polyethylene, biocompatible polymers, and other materials known in the prosthetic arts.

Depending on the patient's disease state, the condition of the patient's natural facet joint-including the facet joint's strength, location and orientation-may not be acceptable. As shown in FIGS. 4-7, therefore, the natural cephalad and caudal facet joint surfaces have been removed to enable the installation of a prosthetic facet joint without limitations presented by remaining portions of the natural facet joint.

In one embodiment of the invention, fixation element 42 attaches cephalad prosthesis 36 to a vertebra 60 in an orientation and position that places bearing surface 40 in approximately the same location as the natural facet joint surface the prosthesis replaces. The prosthesis may also be placed in a location other than the natural facet joint location without departing from the invention, such as by orienting the fixation element along a different angle, by moving the joint cephalad or caudad, or by moving the joint medially or laterally.

In the embodiment shown in FIGS. 4-7, fixation element 42 is a screw. Other possible fixation elements include headless screws, stems, corkscrews, wire, staples, adhesives, bone cements, and other materials known in the prosthetic arts.

In this embodiment of the invention, the cephalad facet joint prosthesis attaches to a posterior element of the vertebra, such as one or portions of the lamina and/or the spinous process. For example, as shown in FIGS. 4-6, fixation element 42 may extend through a lamina portion 62 of vertebra 60 at the base of spinous process 64, traversing the vertebra midline as defined by the spinous process 64 and through another lamina portion 66. This orientation of the fixation element is similar to that used in translaminar facet joint screw fixation, as known in the art. Other orientations of fixation element 42 are possible, of course, depending on the dictates of the specific vertebral anatomy and the desires of the clinician. For example, fixation element 42 may extend through only one lamina portion, only through the spinous process, etc.

Unlike other facet joint prostheses that attach to the pedicle, this embodiment's use of one or more posterior elements of the vertebra to attach the cephalad facet joint prosthesis of this invention does not block access to the pedicle area, leaving this area free to be used to attach other prostheses or devices. Other embodiments of the invention may block the pedicle area, of course, without departing from the scope or spirit of the invention. In addition, because of the inherent strength of the lamina, the cephalad facet joint prosthesis may be affixed without the use of bone cement, especially when using a bone ingrowth surface or trabecular metal.

In the orientation shown in FIGS. 4-6 as well as in some alternative embodiments, after insertion the fixation element's proximal end 43 (preferably formed to mate with a suitable insertion tool) and distal end 44 lie on opposite sides of the lamina. Bearing element 38 attaches to the distal end 44 of fixation element 42 to be disposed between a caudal facet joint bearing surface (either natural or artificial, such as the artificial caudal facet joint prosthesis described below) and a portion of the vertebra, such as the lamina portion shown in FIGS. 4-6. To attach bearing element 38 to fixation element 42 in the embodiment shown in FIG. 4, a hole 46 in bearing element 38 is formed with a Morse taper that mates with the distal end 44 of fixation element 42. Other means of attaching bearing element 38 to fixation element 42 may be used, of course, such as other Morse or other taper connections, machine screw threads, NPT screw threads or other known mechanical fastening means. Fixation element 42 may be coated with antimicrobial, antithrombotic, hydroxyapatite, or osteoinductive materials to promote bone ingrowth and fixation. Bearing element 38 may be attached to fixation element 42 before or after implantation in the patient, depending on the manner of implantation and the requirements of the situation.

Prosthesis 36 may be used to form the cephalad portion of a facet joint with either a natural caudal facet joint portion or an artificial caudal facet joint prosthesis.

FIGS. 4-7 also show an artificial caudal joint prosthesis 50 for replacing the superior half of a natural facet joint according to one aspect of this invention. Caudal prosthesis 50 has a bearing element 52 with a bearing surface 54. In this embodiment, bearing surface 54 is concave. Bearing element 52 may be formed from biocompatible metals (such as cobalt chromium steel, surgical steels, titanium, titanium alloys, tantalum, tantalum alloys, aluminum, etc.), ceramics, polyethylene, biocompatible polymers, and other materials known in the prosthetic arts, and bearing surface 54 may be formed from biocompatible metals (such as cobalt chromium steel, surgical steels, titanium, titanium alloys, tantalum, tantalum alloys, aluminum, etc.), ceramics, polyethylene, biocompatible polymers, and other materials known in the prosthetic arts.

In one embodiment, the natural caudal facet surface has been removed, and fixation element 56 attaches prosthesis 50 to a vertebra 70 via a pedicle in an orientation and position that places bearing surface 54 in approximately the same location as the natural facet joint surface the prosthesis replaces. In an alternative embodiment, the bearing surface 54 may be placed in a location different than the natural facet joint surface, either more medial or more lateral, more cephalad or more caudad, and/or rotated from the natural anatomical orientation and orientation. In addition, in other embodiments the caudal component can be attached to the vertebral body in addition to the pedicle or to the vertebral body alone.

As shown in the embodiment of FIGS. 4-7, fixation element 56 is a screw attached to bearing element 54 via a hole 58 formed in bearing element 52 and is inserted into a pedicle portion 72 of vertebra 70. Other possible fixation elements include stems, corkscrews, wire, staples, adhesives, bone cements, and other materials known in the prosthetic arts. Fixation element 56 can also be inserted into the vertebral body in addition to or in place of the pedicle.

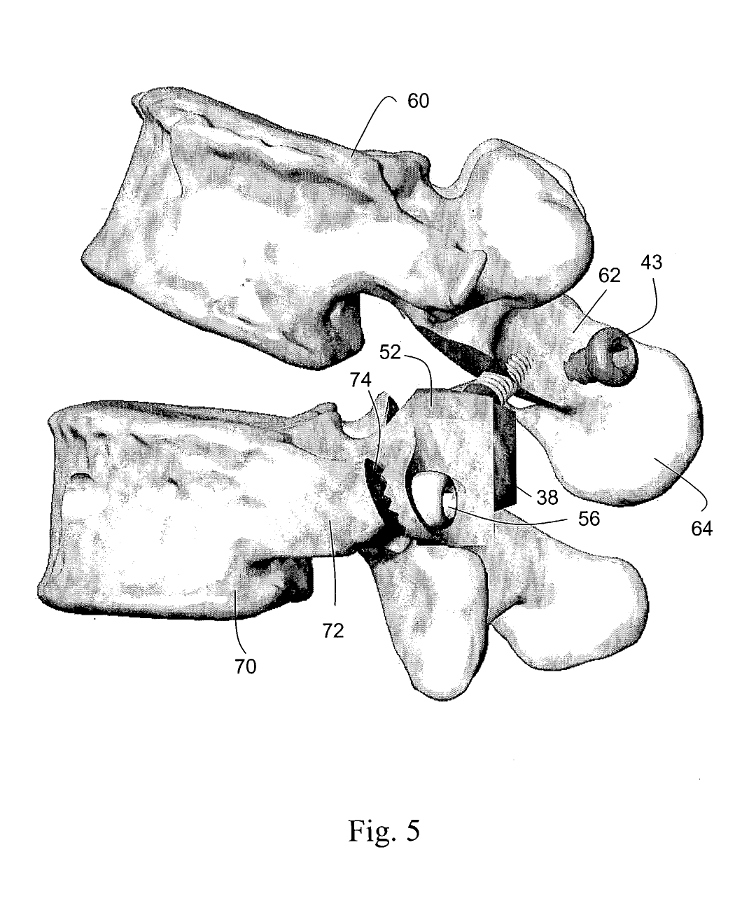

In this embodiment, bearing element 52 has a serrated fixation surface 57 adapted to contact a contact portion 74 of vertebra 70. This optional fixation surface 57 helps prevent rotation of the bearing element 52. In addition, fixation surface 57 may be coated with bone ingrowth material, and any optional serrations increase the surface area for bone ingrowth. As shown in FIG. 5, in this embodiment the entire bearing surface 54 is posterior to surface 57 and contact portion 74.

Prosthesis 50 may be used to form the caudal portion of a facet joint with either a natural cephalad facet joint portion or an artificial cephalad facet joint prosthesis.

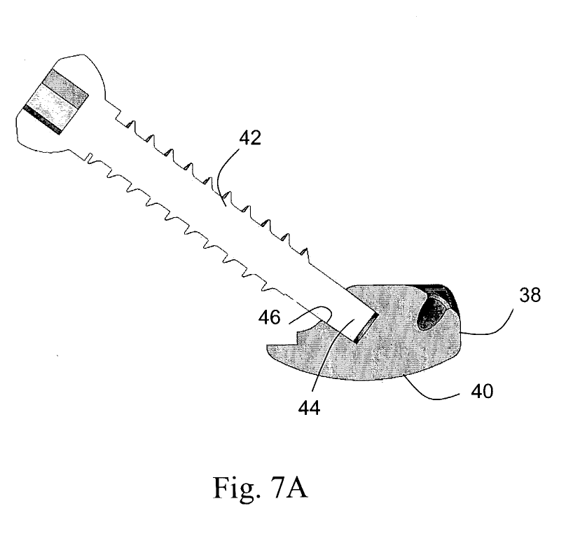

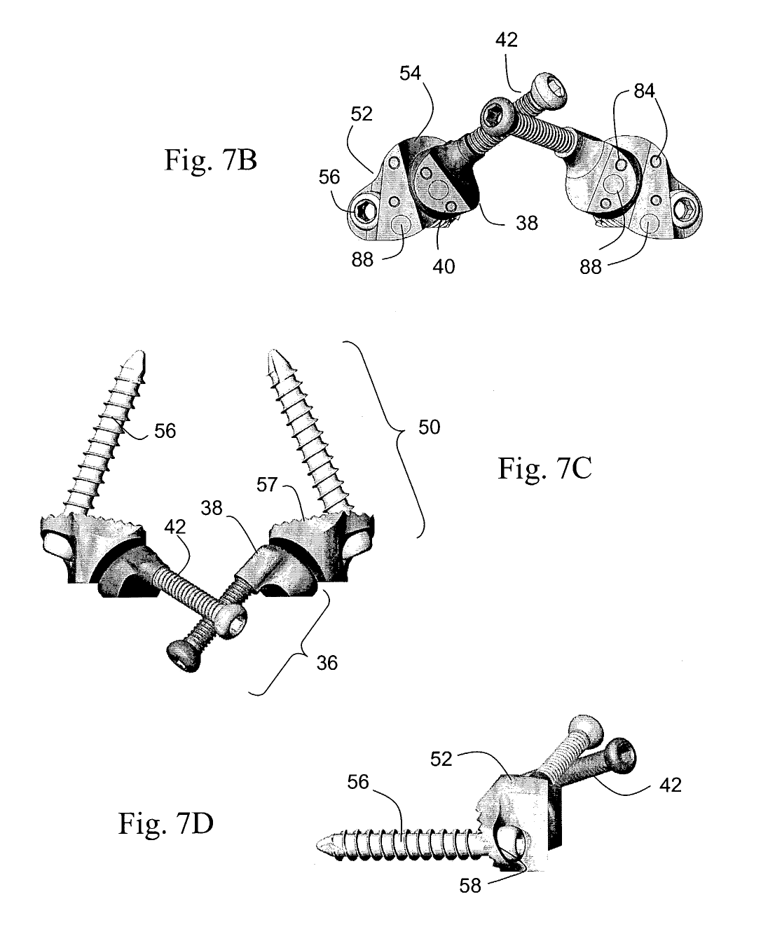

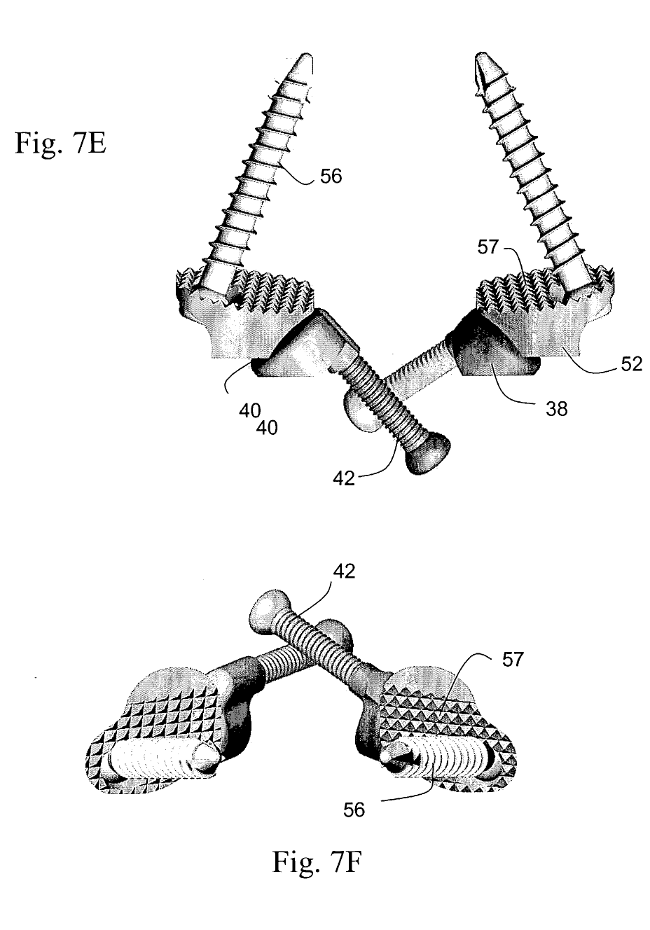

FIGS. 7A-F show the artificial facet joint prosthesis according to one embodiment of this invention apart from the vertebrae. As shown, cephalad bearing surface 40 and caudal bearing surface 54 meet to form an artificial facet joint. As seen best in FIG. 7B, the width of caudal bearing surface 54 along its transverse axis is greater than the width of cephalad bearing surface 40 along its transverse axis. This feature helps align the cephalad and caudal joints during implant. In addition, this feature permits the point of contact between the two bearing surface to change with flexion, extension, left and right rotation and lateral bending of the patient's spine.

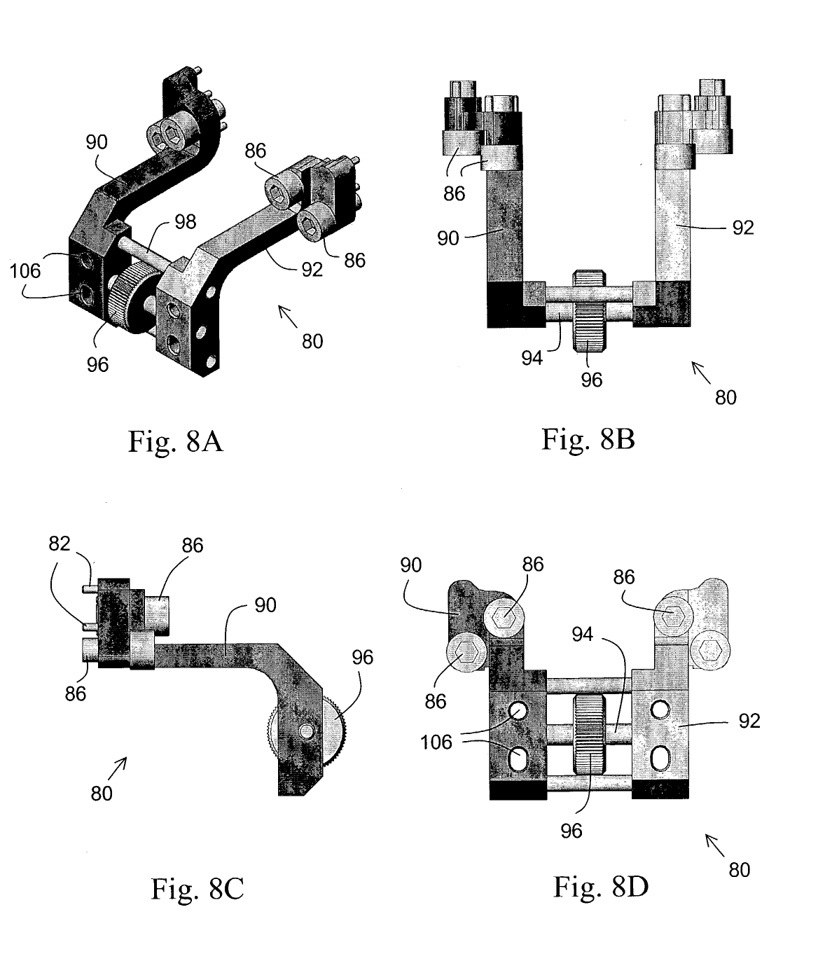

The prostheses of FIGS. 4-7 may be implanted without special tools. One embodiment of the invention, however, includes an installation fixture to assist with the implantation procedure. FIGS. 8-14 show installation tools used to implant two artificial facet joints, i.e., two cephalad facet joint prostheses and two corresponding caudal facet joint prostheses. The invention also includes installation tools for implanting a single facet joint prosthesis, two caudal facet joint prostheses, two cephalad facet joint prostheses, a caudal and cephalad joint prosthesis, or any other combination of facet joint prostheses.

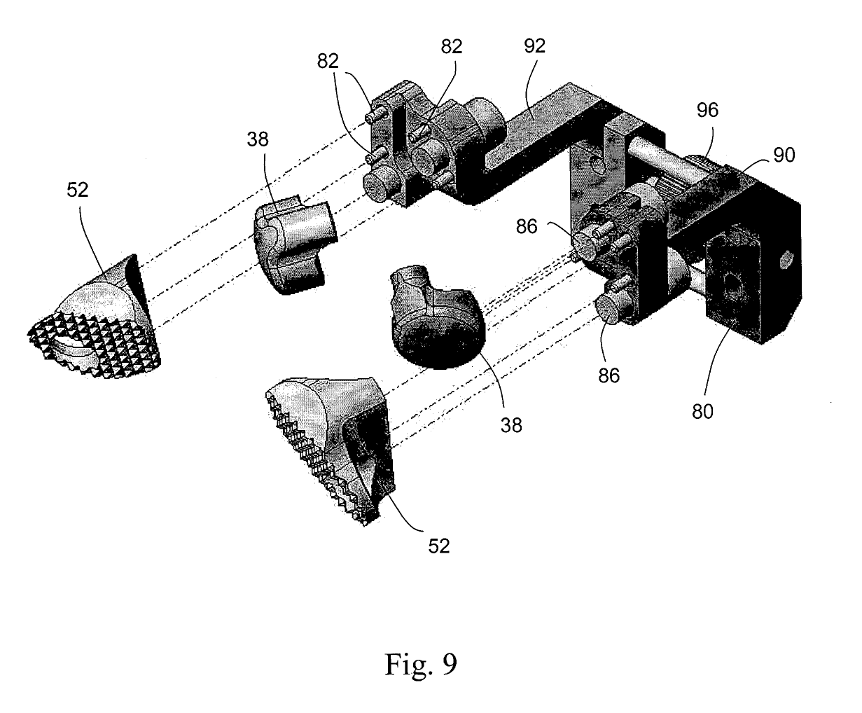

As shown in FIGS. 8 and 9, installation fixture 80 has alignment elements 82 to align the cephalad bearing elements 38 and caudal bearing elements 52. In this embodiment, the alignment elements are two dowels for each bearing element. Alignment elements 82 mate with corresponding alignment elements in the bearing elements, such as holes 84 (shown, e.g., in FIG. 7B) formed in cephalad bearing elements 38 and caudal bearing elements 52. Other alignment elements may be used, of course, such as pins, grooves, indentations, etc. Attachment elements such as screws 86 attach the bearing elements 38 and 52 to the installation fixture via screw holes 88 (shown, e.g., in FIG. 7B) formed in the bearing elements and in installation fixture 80.

When attached to installation fixture 80, cephalad and caudal bearing surfaces 40 and 54 are in contact and in proper alignment with respect to each other, as shown in FIG. 8. In one embodiment, the cephalad and caudal bearing surfaces 40 and 54 are preloaded to be in compression when attached to installation fixture 80. To bring the pairs of bearing surfaces in proper alignment with respect to the patient's vertebrae, the spacing between the pairs of bearing surfaces might need to be adjusted. In the embodiment of FIGS. 8, 9 and 11-14, installation fixture 80 has two bearing support components 90 and 92 that move in a controlled manner with respect to each other. Specifically, in this embodiment a threaded shaft 94 extends between support components 90 and 92. Shaft 94 engages bores formed in support components 90 and 92; one or both of the bores are threaded so that rotation of shaft 94 causes support components 90 and 92 to move towards or away from each other. Shaft 94 may be provided with a thumbwheel 96 or other actuator for ease of use. One or more guide rods 98 may be provided to maintain the alignment of support components 90 and 92. Other means of moving the cephalad/caudal bearing elements pairs with respect to each other may be used, such as a guided or unguided sliding connection between installation fixture elements.

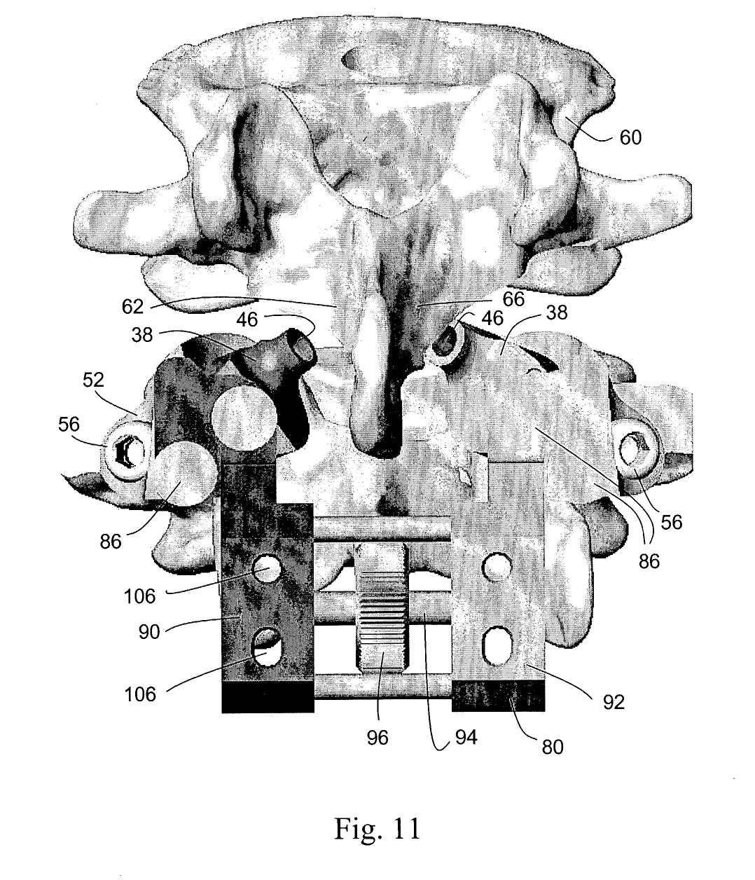

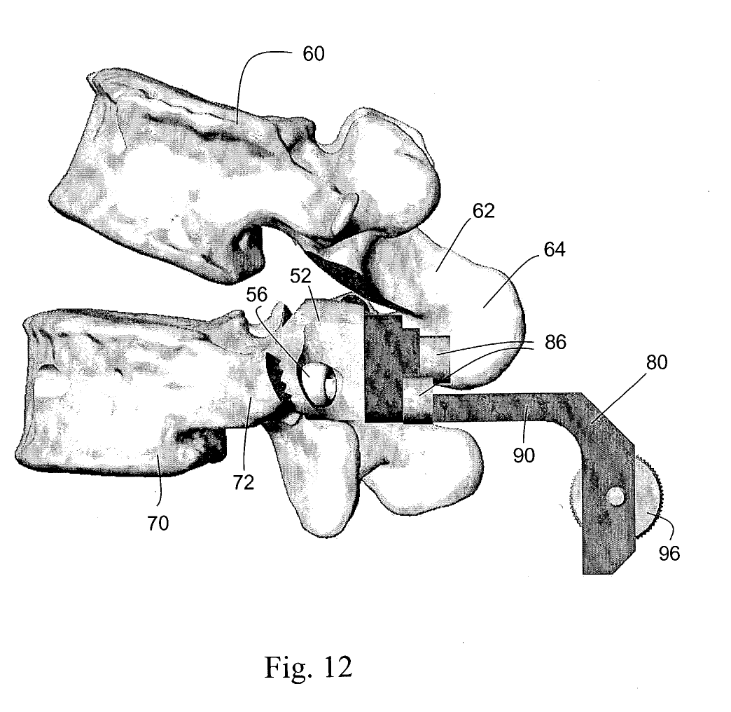

In use, after preparing the implant site by removal of all or a portion of existing natural cephalad and caudal facet joint portions of the cephalad and caudal vertebrae 60 and 70, respectively, of the spine motion segment, bearing elements 38 and 52 are attached to installation fixture 80 as described above. The spacing between the bearing element pairs is then adjusted using thumbwheel 96 to align the fixation holes 58 of caudal bearing elements 52 with the proper fixation screw insertion sites in the pedicle portions of the caudal vertebra (or other suitable location), thus placing the artificial facet joints in positions corresponding to the position of natural facet joints or in any other position desired by the physician, including positions that do not correspond to the position of natural facet joints. Passages aligning with holes 58 are formed and in the pedicle—or into another part of the caudal vertebra near or adjacent to the pedicle—using a drill, awl, pedicle probe, or other tool known in the surgical arts. Fixation screws 56 are then inserted through holes 58 into the pedicle or other portion of the caudal vertebra to attach the caudal bearing elements as well as the entire prosthesis and installation fixture to the caudal vertebra 70, as shown in FIGS. 11 and 12. Alternatively, self-tapping screws or other caudal fixation elements may be used, thereby eliminating the need to pre-form the passages.

Thereafter, the cephalad bearing elements are attached to the cephalad vertebra 60. In one embodiment, an insertion path is first determined for each fixation element, then a passage is formed along the insertion path corresponding to cephalad bearing element holes 46 (e.g., in the lamina at the base of the spinous process and through the lamina on the other side, through only one lamina portion, through the spinous process, etc.). Fixation screws 42 can then be inserted through the holes 46 into the passages. Alternatively, self-tapping screws or other caudal fixation elements may be used, thereby eliminating the need to pre-form the passages.

After all four bearing elements have been affixed, the installation fixture 80 may be detached and removed. Installation fixture 80 may be used to implant fewer than four bearing elements, of course.

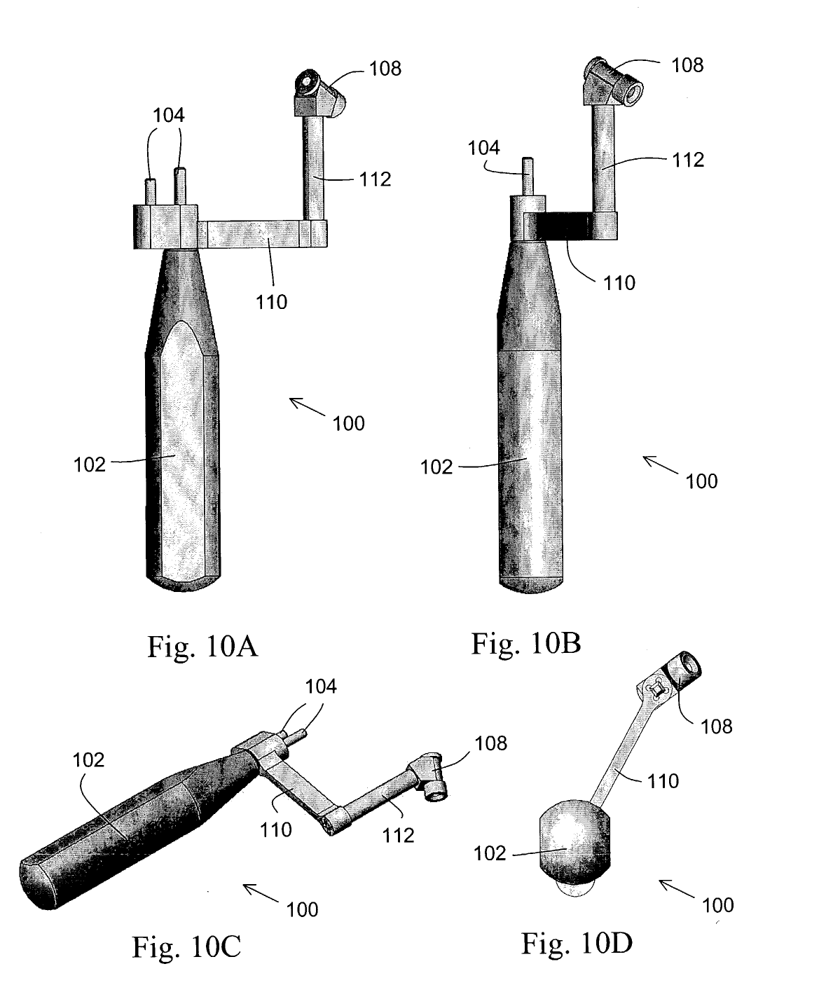

FIGS. 10, 13 and 14 show a tool that may be used to define the insertion path (location, orientation, etc.) for the fixation element of the left cephalad bearing element. For example, the tool may be used to guide the formation of a cephalad bearing element attachment passage for the left bearing element. A corresponding mirror image tool may be used for the right cephalad bearing element. In alternative embodiments, a single tool may be used for defining the insertion path for both left and right cephalad bearing elements.

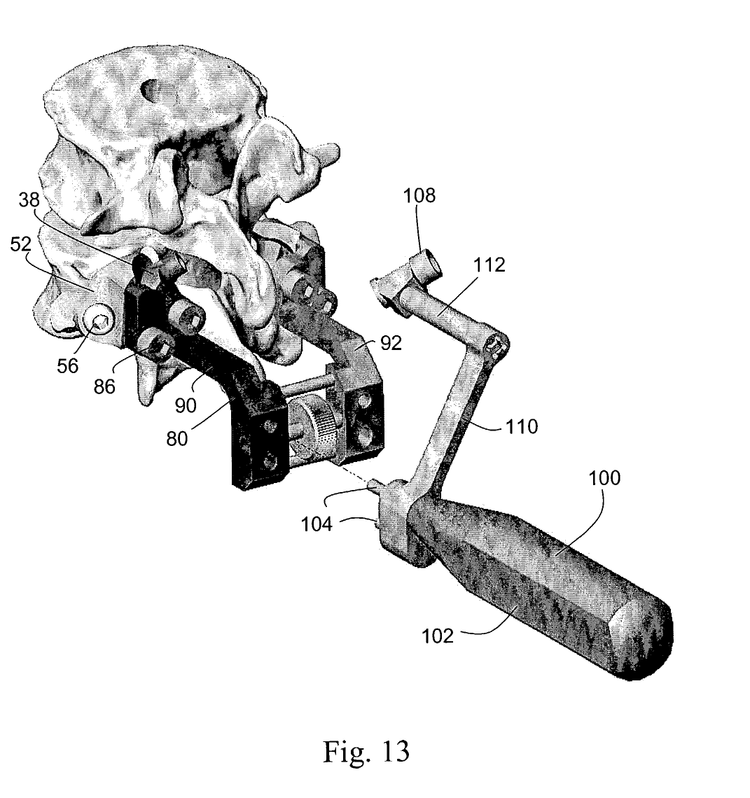

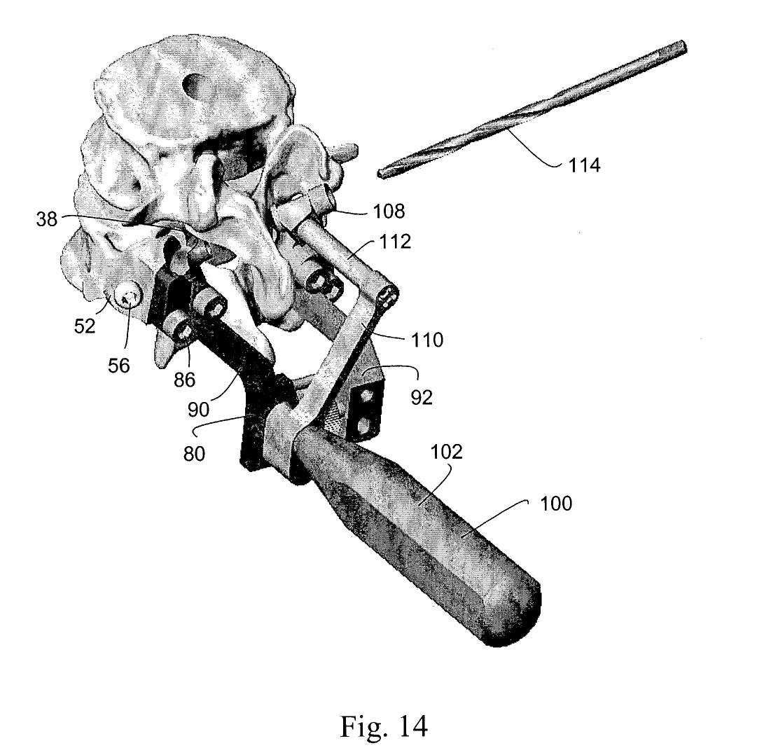

As shown, tool 100 has a handle 102 and an alignment interface (such as dowels 104 in tool 100 and holes 106 in fixture 80) to align the tool in the proper orientation with respect to installation fixture 80 and a cephalad facet joint bearing element. With the caudal and cephalad bearing elements still attached to installation fixture 80 and preferably with caudal bearing elements already affixed to the caudal vertebra 70, tool 100 engages installation fixture through the alignment interface as shown in FIGS. 13 and 14. In this position, tool 100 may be used to define an insertion path for the cephalad fixation elements.

In the embodiment shown in FIGS. 10, 13 and 14, the insertion path guide is a drill guide 108 supported by arms 110 and 112 and is aligned with hole 46 in cephalad bearing element 38 by the alignment interface between installation fixture 80 and guide tool 100. In this embodiment, drill guide 108 is a tube, but other guide elements may be used, such as a guide groove or surface. A drill bit 114 may be inserted through drill guide 108 to form an insertion passage, such as a passage through a lamina portion of the cephalad vertebra. A fixation screw may then be inserted through the passage in the cephalad vertebra and into the Morse taper connection of hole 46 (or other type connection, as discussed above) of cephalad bearing element 38. As discussed above, the fixation screw may be coated with a bone ingrowth material. Alternatively, a self-tapping screw may be used, thereby removing the need to pre-form a passage.

A mirror image tool may then be used to define an insertion path or to form a hole for the right cephalad bearing element, which is then affixed to the vertebral body in the same way. The installation fixture is then removed, such as by unscrewing screws 86.

As mentioned above, in alternative embodiments the guide tool may be used to define a path for a self-tapping screw or other fixation element that does not require the use of a drill. In those embodiments, element 108 may be used to define a path for the self-tapping screw or other fixation element. The fixation element path may be through only a single lamina portion, through the spinous process alone, or any other suitable path.

In some embodiments, the entire prosthesis other than the bearing surface may be coated with bone ingrowth material.

The above described embodiments of this invention are merely descriptive of its principles and are not to be limited. The scope of this invention instead shall be determined from the scope of the following claims, including their equivalents.

What is claimed is:

1. A tool for installing a facet joint prosthesis, the tool comprising:
right and left caudal facet joint bearing element attachment mechanisms; and
a caudal facet joint bearing element spacing adjuster adapted and configured to change the spacing between right and left caudal facet joint bearing element attachment mechanisms.

2. The tool of claim 1 wherein the caudal facet joint bearing element spacing adjuster comprises an actuator.

3. The tool of claim 1 further comprising right and left caudal facet joint bearing element alignment elements adapted and configured to mate with corresponding alignment elements on caudal facet joint bearing elements.

4. The tool of claim 1 further comprising right and left cephalad facet joint bearing element attachment mechanisms oriented with the right and left caudal facet joint bearing element attachment mechanisms such that a right cephalad facet joint bearing element mounted in the right cephalad facet joint bearing element attachment mechanism aligns with a right caudal facet joint bearing element mounted in the right caudal facet joint bearing element attachment mechanism and a left cephalad facet joint bearing element mounted in the left facet joint bearing element attachment mechanism aligns with a left caudal facet joint bearing element mounted in the left caudal facet joint bearing element attachment mechanism.

5. The tool of claim 4 wherein the right and left cephalad facet joint bearing element attachment mechanisms and the right and left caudal facet joint bearing element attachment mechanisms are adapted and configured to orient the right and left cephalad facet joint bearing elements with the right and left caudal facet joint bearing elements prior to contacting the facet joint prosthesis with a vertebra.

6. The tool of claim 4 wherein the caudal facet joint bearing element spacing adjuster is further adapted and configured to change the spacing of the right and left cephalad facet joint bearing elements.

7. The tool of claim 6 wherein the right cephalad facet joint bearing element attachment mechanism is affixed to the right caudal facet joint bearing element attachment mechanism and the left cephalad facet joint bearing element attachment mechanism is affixed to the left caudal facet joint bearing element attachment mechanism.

8. The tool of claim 4 further comprising right and left cephalad facet joint bearing element alignment elements adapted and configured to mate with corresponding alignment elements on cephalad facet joint bearing elements.

9. The tool of claim 4 further comprising an insertion path guide interface adapted and configured to orient a fixation element insertion path guide in a specific orientation with respect to at least one of the right and left cephalad facet joint bearing surfaces.

10. The tool of claim 4 further comprising an insertion path guide interface adapted and configured to orient a fixation element insertion path guide to guide the orientation of a cephalad facet joint fixation element in a lamina portion of a vertebra.

11. The tool of claim 4 further comprising an insertion path guide interface adapted and configured to orient a fixation element insertion path guide to guide the attachment of a cephalad facet joint bearing element to a vertebra without blocking access to a pedicle portion of the vertebra.

12. The tool of claim 4 further comprising right and left insertion path guide interfaces each adapted and configured to orient a fixation element insertion path guide in a specific orientation with respect to the right and left cephalad facet joint bearing surfaces, respectively.

13. The tool of claim 4 further comprising right and left insertion path guide interfaces each adapted and configured to orient a fixation element insertion path guide to guide the insertion path of a cephalad facet joint fixation element in a lamina portion of a vertebra.

14. The tool of claim 4 further comprising right and left insertion path guide interfaces each adapted and configured to orient a fixation element insertion path guide to guide the attachment of a cephalad facet joint bearing element to a vertebra without blocking access to pedicle portions of the vertebra.

15. A tool for installing a facet joint prosthesis, the tool comprising:
a caudal facet joint bearing surface attachment mechanism;
a cephalad facet joint bearing surface attachment mechanism adapted and configured to align a cephalad joint bearing surface with a caudal facet joint bearing surface mounted in the caudal facet joint bearing surface attachment mechanism; and
an insertion path guide interface.

16. The tool of claim 15 wherein the insertion path guide interface is adapted and configured to orient a fixation element insertion path guide in a specific orientation with respect to a cephalad facet joint bearing surface.

17. The tool of claim 15 wherein the insertion path guide interface is adapted and configured to orient a fixation element insertion path guide to guide the orientation of a cephalad facet joint fixation element in a lamina portion of a vertebra.

18. The tool of claim 15 wherein the insertion path guide interface is adapted and configured to orient a fixation element insertion path guide to guide the attachment of a cephalad facet joint bearing element to a vertebra without blocking access to a pedicle portion of the vertebra.

19. The tool of claim 15 wherein the caudal facet joint bearing surface attachment mechanism and the cephalad facet joint bearing surface attachment mechanism are further adapted and configured to place the caudal facet joint bearing surface and the cephalad facet joint bearing surface in compression.

20. A guide tool for guiding the insertion path of a fixation element for a cephalad facet joint bearing element of a facet joint prosthesis, the guide tool comprising:
a handle;
a facet joint bearing element alignment interface; and
an insertion path guide surface.

21. The guide tool of claim 20 wherein the handle has a handle axis, the guide tool further comprising an arm extending from the handle and being adapted and configured to orient the insertion path guide surface along an axis other than the handle axis.

22. The guide tool of claim 21 wherein the arm comprises a first section extending perpendicularly from the handle axis and a second section extending from the first section parallel with the handle axis.

23. The guide tool of claim 20 wherein the handle has a handle axis, the guide tool further comprising an arm extending from the handle and being adapted and configured to orient the insertion path guide surface along an axis extending through a lamina portion of the vertebra when the handle axis is oriented substantially perpendicular to a central axis of a vertebra.

24. The guide tool of claim 20 wherein the insertion path guide surface comprises a tube.

25. The guide tool of claim 20 wherein the insertion path guide surface is adapted and configured to accommodate a drill bit.

26. The guide tool of claim 20 wherein the facet joint bearing surface alignment interface comprises protrusions extending from the guide tool.

27. A facet joint prosthesis installation assembly comprising:
a caudal facet joint bearing surface attachment mechanism;
a cephalad facet joint bearing surface attachment mechanism adapted and configured to align a cephalad joint bearing surface with a caudal facet joint bearing surface mounted in the caudal facet joint bearing surface attachment mechanism;
a guide tool for guiding the insertion path of a fixation element for a cephalad facet joint bearing surface of the facet joint prosthesis; and
a guide tool interface adapted and configured to orient the guide tool to guide the insertion path of a cephalad facet joint bearing surface fixation element in a vertebra.

28. The assembly of claim 27 wherein the guide tool interface is further adapted and configured to orient the guide tool to guide the insertion path of the cephalad facet joint bearing surface fixation element in a lamina portion of the vertebra.

29. The assembly of claim 27 wherein the guide tool interface is further adapted and configured to orient the guide tool to guide the insertion path of the cephalad facet joint bearing surface fixation element to the vertebra without blocking access to a pedicle portion of the vertebra.

30. The assembly of claim 27 wherein the caudal facet joint bearing surface attachment mechanism comprises a right caudal facet joint bearing surface attachment mechanism and the cephalad facet joint bearing surface attachment mechanism comprises a right cephalad facet joint bearing surface attachment mechanism, the assembly further comprising a left caudal facet joint bearing surface attachment mechanism and a left cephalad facet joint bearing surface attachment mechanism adapted and configured to align a cephalad joint bearing surface with a caudal facet joint bearing surface mounted in the left caudal facet joint bearing surface attachment mechanism.

31. The assembly of claim 30 further comprising an actuator adapted and configured to change the spacing between the right and left caudal facet joint bearing surface attachment mechanisms.

32. The assembly of claim 31 wherein the actuator is further adapted and configured to changed the spacing between the right and left cephalad facet joint bearing surface attachment mechanisms.

33. The assembly of claim 30 wherein the guide tool interface is a first guide tool interface, the assembly further comprising a second guide tool interface adapted and configured to orient the guide tool to guide the insertion path of a cephalad facet joint bearing surface fixation element in the vertebra.

34. The assembly of claim 30 wherein the guide tool is a first guide tool and the guide tool interface is a first guide tool interface, the assembly further comprising a second guide tool and a second guide tool interface adapted and configured to orient the second guide tool to guide the insertion path of a cephalad facet joint bearing surface fixation element in the vertebra.

35. The assembly of claim 27 wherein the caudal facet joint bearing surface attachment mechanism and the cephalad facet joint bearing surface attachment mechanism are further adapted and configured to place the caudal facet joint bearing surface and the cephalad facet joint bearing surface in compression.

36. The assembly of claim 27 wherein the guide tool comprises a handle and an insertion path guide surface.

37. The assembly of claim 36 wherein the handle has a handle axis, the guide tool further comprising an arm extending from the handle and being adapted and configured) to orient the insertion path guide surface along an axis other than the handle axis.

38. The assembly of claim 37 wherein the arm comprises a first section extending perpendicularly from the handle axis and a second section extending from the first section parallel with the handle axis.

39. The assembly of claim 36 wherein the handle has a handle axis, the guide tool further comprising an arm extending from the handle and being adapted and configured to orient the insertion path guide surface along an axis extending through a lamina portion of the vertebra when the handle axis is oriented substantially perpendicular to a central axis of the vertebra.

40. The assembly of claim 36 wherein the insertion path guide surface comprises a tube.

41. The assembly of claim 36 wherein the insertion path guide surface is adapted and configured to accommodate a drill bit.

42. A method for implanting a cephalad facet joint prosthesis to replace a removed cephalad portion of a natural facet joint on a vertebra, the method comprising the steps of:
placing the cephalad facet joint prosthesis in a desired position with respect to the vertebra;
aligning a guide tool with the cephalad facet joint prosthesis; and
using the guide tool to guide an insertion path for a cephalad facet joint fixation element.

43. The method of claim 42 wherein the placing step comprises placing the cephalad facet joint prosthesis between a caudal facet joint bearing surface and the vertebra.

44. The method of claim 42 wherein the aligning step comprises aligning a guide tool alignment interface with cephalad facet joint prosthesis.

45. The method of claim 42 wherein the guide tool comprises an insertion path guide surface, the using step comprising using the insertion path guide surface to guide the insertion path.

46. The method of claim 42 wherein the using step comprises forming a passage in the vertebra.

47. The method of claim 46 wherein the forming step comprises drilling the passage in the vertebra.

48. The method of claim 42 wherein the using step comprises inserting the cephalad facet joint fixation element.

49. The method of claim 42 wherein the using step comprises using the guide tool to guide an insertion path for the cephalad facet joint fixation element through a lamina portion of the vertebra.
